(19) United States
(12) Patent Application Publication
(10) Pub. No.: US 2026/0069731 A1
(43) Pub. Date: Mar. 12, 2026

(54) DEVICES AND METHODS FOR MEDICAL DEVICE DISINFECTION

(71) Applicant: Mick Abae, Davie, FL (US)

(72) Inventor: Mick Abae, Davie, FL (US)

(21) Appl. No.: 19/392,654

(22) Filed: Nov. 18, 2025

Related U.S. Application Data
(63) Continuation-in-part of application No. 18/242,182, filed on Sep. 5, 2023.
(60) Provisional application No. 63/405,124, filed on Sep. 9, 2022.
(60) Provisional application No. 63/421,420, filed on Nov. 1, 2022.

(51) Int. Cl. A61L 2/10; A61L 2/26

(52) U.S. Cl. CPC A61L 2/10; A61L 2/26; A61L 2103/15; A61L 2202/11; A61L 2202/122; A61L 2202/16

(57) ABSTRACT

A disinfection system for a medical probe is disclosed. The system can include a container configured to receive the medical probe. A plurality of ultraviolet (UV) light emitters can be included for emitting UV light in the container to disinfect a surface of the medical probe. A bracket system can be coupled to the container and configured to couple the container to a portion of a medical device comprising the medical probe or a mobile caster holding pole.

### CROSS-REFERENCE TO RELATED APPLICATIONS

This application is a Continuation-in-Part (CIP) of U.S. patent application Ser. No. 18/242,182, filed on Sep. 5, 2023, which claims the benefit of U.S. Provisional Application No. 63/405,124, filed on Sep. 9, 2022, and U.S. Provisional Application No. 63/421,420, filed on Nov. 1, 2022. The entire disclosures of the foregoing applications are hereby incorporated by reference in their entirety.

### TECHNICAL FIELD

The present application relates to devices and methods for disinfecting a medical device.

### BACKGROUND

The disinfection of medical devices intended for repeated use is a critical factor in ensuring patient safety.

The use of medical devices can present a risk of pathogen transmission between patients if proper cleaning or sterilization procedures are not followed. Different types of medical devices require different levels of disinfection depending on their intended use and the nature of patient contact. Devices that penetrate the skin or mucous membranes (e.g., such as scissors, forceps, tweezers, and hemostats) typically require sterilization. Devices that contact mucous membranes or internal body surfaces, including intracavitary or endocavitary probes such as ultrasound transducers, generally require high-level disinfection rather than full sterilization. In contrast, devices that only contact intact skin without penetrating or contacting mucous membranes (e.g., blood pressure cuffs, stethoscopes, or external transducers) generally require only low-level or intermediate disinfection.

Medical probes that directly contact internal body surfaces or mucous membranes, for example intracavitary ultrasound probes, demand high-level disinfection and the use of high-quality, single-use protective covers for each procedure. Adherence to proper high-level disinfection protocols is essential to prevent cross-contamination and ensure patient safety.

Embodiments of the current disclosure address some of the above-described limitations. The scope of the current disclosure, however, is defined by the attached claims, and not by the ability to solve any specific problem.

### SUMMARY

This Summary is provided to introduce a selection of concepts in simplified form that are further described below in the Detailed Description. This Summary is not intended to identify key aspects or essential aspects of the claimed subject matter.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

As described herein, an aspect of the present disclosure relates to a disinfection device for a medical probe or a method of disinfecting a medical probe.

In some examples, a disinfection system for a medical probe is disclosed. The system can include a container configured to receive at least a portion of the medical probe and a plurality of ultraviolet (UV) light emitters arranged to emit UV light within the container to disinfect an external surface of the medical probe. A bracket system can be coupled to the container and configured to couple the container to a portion of a medical device that houses or interfaces with the probe, such as an ultrasound machine, or to a mobile caster holding pole.

In some examples, the system includes a probe holder positioned within the container to hold the medical probe apart from the plurality of UV light emitters to provide a standoff distance that promotes dispersion of UV light over the probe surface when at least a portion of the medical probe is inserted into the container and the probe holder.

In some examples, the medical device includes an ultrasound machine having a base and a control housing configured to store one or more probes, including, for instance, an endoprobe and an external probe.

In some examples, the bracket system can be configured as a detachable bracket that supports the container and attaches to an edge of the medical device (e.g., an ultrasound machine).

In some examples, the bracket system includes a clamp mount with opposed upper and lower clamping surfaces configured to engage opposite sides of the edge, the clamp mount optionally including a tightening mechanism such as a threaded screw, a lever-actuated cam, or a quick-release fastener.

In some examples, the bracket system includes a coupler extending from the clamp to the container, the coupler being detachable or interchangeable.

In some examples, the system is configured for tool-free installation and removal from the medical device.

In some examples, the container and bracket system can be formed from sterilizable or disinfectant-compatible materials and can be configured to support an endoprobe simultaneous with a receptacle of the medical device supporting one or more other probes.

In some examples, the container includes a safety cap configured to connect to and seal an opening of the container, the safety cap including a UV-blocking material and a gasket to reduce or prevent UV light leakage from the container during operation.

In some examples, the bracket system includes a multi-part bracket assembly including a first bracket member having at least one female groove and a counterpart second bracket member having at least one corresponding male groove configured to slide into and out of the female groove to provide a detachable sliding engagement.

In some examples, a beam extends outwardly from an outer surface of the container and includes a rail profile, such as a dovetail, a T-slot, or a rectangular tongue, the beam being integrally formed with or detachably connectable to the container.

In some examples, the first bracket member can include a plurality of female grooves arranged in parallel and the second bracket member a corresponding plurality of male grooves arranged in parallel to provide a selectable height or position of the container relative to the medical device.

In some examples, the bracket system is configured such that the container and a connector beam of the container are slidable in a direction substantially parallel to an edge of the medical device to transition between a disengaged position and a secured position.

In some examples, the bracket system includes a proximal portion pivotably connected to a distal portion. A hinge can connect the proximal portion to the distal portion, the hinge including at least one of a pin joint, a living hinge, and a friction hinge configured to maintain a selected angle between portions and to permit pivoting while maintaining alignment of grooves or mating profiles for sliding engagement with the container.

In some examples, in an unconnected configuration the distal portion is oriented angled downward relative to the proximal portion, and in a connected configuration the distal portion is pivotable to an orientation in which the proximal and distal portions are substantially parallel.

In some examples, the distal portion can include one or more male grooves configured to detachably connect with corresponding female grooves of a beam extending from the container, or one or more female grooves configured to detachably connect with corresponding male grooves of a beam extending from the container.

In some examples, a bracket system is provided that includes a proximal portion with a mounting interface configured to couple to a medical device or to a mobile caster holding pole, and a distal portion including a container interface configured to couple to the container by sliding engagement.

In some examples where the pole includes a caster-mounted base and a central vertical rod, the mounting interface includes a clamp configured to engage an outer surface of the pole, optionally including a tightening mechanism such as a threaded screw, a lever-actuated cam, or a quick-release fastener.

In some examples, a method of disinfecting a medical probe is disclosed. The method can include inserting at least a portion of the medical probe through a container containing a plurality of ultraviolet (UV) light emitters, the container being detachably coupled to a medical device by using a bracket system; inserting at least a portion of the medical probe into a probe holder to hold the medical probe apart from the plurality of UV light emitters to provide a distance to disperse UV light over a surface of the medical probe; and disinfecting the surface of the medical probe using at least the plurality of UV light emitters.

In some examples, the bracket system includes a first bracket member with at least one female groove and a second bracket member with at least one corresponding male groove.

In some examples, the method includes detachably coupling the container to the medical device by sliding the male groove into the female groove.

In some examples, the sliding the male groove into the female groove includes sliding a beam extending outwardly from an outer surface of the container into the female groove of the bracket system.

In some examples, the method includes sliding the male and female grooves relative to one another to transition between a disengaged position and a secured position of the container relative to the medical device.

In some examples, the bracket system including a first bracket member with at least one female groove and a second bracket member with at least one corresponding male groove.

In some examples, the method includes pivoting a distal portion of the bracket system relative to a proximal portion of the bracket system.

In some examples, the bracket system includes a hinged connector including an unconnected configuration where the distal portion is angled downward relative to the proximal portion.

In some examples, the pivoting the distal portion includes, in a connected configuration, pivoting the distal portion to an orientation in which the proximal and distal portions are substantially parallel.

In some examples, the method includes coupling the distal portion to the beam by sliding engagement between grooves on the distal portion and corresponding grooves on the beam.

In some examples, the distal portion includes one or more male grooves and the beam includes corresponding female grooves.

In some examples, the distal portion includes one or more female grooves and the beam includes corresponding male grooves.

In some examples, the method includes locking the distal portion in the substantially parallel orientation during disinfecting and unlocking the distal portion to permit pivoting during removal.

In some examples, the method includes positioning an endoprobe within the container such that the endoprobe is supported in a stable orientation relative to the medical device.

In some examples, the method includes articulating the container between a loading orientation for probe insertion and an operating orientation for disinfecting while maintaining groove alignment for sliding engagement with the bracket system.

In some examples, the method includes supplying power to the plurality of UV light emitters to emit the UV light to disinfect the surface of the medical probe.

In some examples, the method includes attaching a first end of the bracket system to the medical device; and attaching the container to a second of the bracket system opposite the first end.

In some examples, the method includes attaching a first end of the bracket system to the medical device by engaging a clamp mount to an edge portion of a base of the medical device, the clamp mount including a tightening mechanism to draw opposed upper and lower clamp surfaces into frictional contact with the base.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE FIGURES

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 illustrates an embodiment of a device to disinfect a medical probe;

FIG. 2 illustrates an embodiment of a device having a holder to hold a plurality of medical probes to disinfect the plurality of medical probes;

FIG. 3 illustrates a perspective view of a bracket-mounted disinfection container attached to an example ultrasound system according to one example;

FIG. 4 illustrates a side profile view of an exemplary container connected to the bracket system according to one example;

FIGS. 5A and 5B are side profile views of an exemplary bracket system attached to exemplary container;

FIG. 6A illustrates a perspective view of another bracket-mounted disinfection container attached to an example ultrasound system according to one example;

FIG. 6B illustrates a close-up partial view of the bracket system of FIG. 6A in a disconnected, unhinged state according to one example;

FIG. 6C illustrates a similar close-up partial view of the bracket system of FIGS. 6A and 6B in a connected, hinged state according to one example;

FIG. 7 illustrates a perspective view of another bracket system mounted to a mobile caster system associated with an ultrasound system according to one example;

FIG. 8 is a flow diagram for a method of disinfecting a medical probe according examples of this disclosure;

FIG. 9 is a flow diagram for a method of disinfecting a medical probe according examples of this disclosure.

### DETAILED DESCRIPTION

A detailed description of one or more embodiments of the present invention is provided below, together with accompanying figures that illustrate the principles of the invention. Although the invention is described in connection with specific embodiments, it is not limited thereto. The scope of the invention is defined solely by the appended claims. Numerous specific details are included herein to provide a thorough understanding of the invention; however, the invention may be practiced without some or all of these details. For clarity, technical material known to those skilled in the relevant art has not been described in unnecessary detail to avoid obscuring the invention.

Unless otherwise defined, all technical and scientific terms used herein have the same meaning as commonly understood by one of ordinary skill in the art to which this invention pertains. As used herein, and unless stated otherwise or required by context, each of the following terms shall have the definitions provided below.

As used herein, the term “patient” refers to an individual whose body or a portion thereof, such as a body surface or internal tissue, is to be contacted by a medical device, such as a medical probe.

As used herein, the term “medical probe” refers to any diagnostic or therapeutic device intended to contact a patient's body surface or internal tissue, including but not limited to ultrasound probes, transducers, transvaginal probes, transrectal probes, intraoperative probes, and transesophageal probes.

As used herein, the term “disinfection” refers to a process that eliminates or reduces pathogenic microorganisms on a surface or object to a level considered safe for reuse, but that does not necessarily destroy all microbial forms such as bacterial spores.

As used herein, the term “high-level disinfection” refers to a disinfection process capable of inactivating all microbial pathogens, including viruses, fungi, and vegetative bacteria, but not necessarily all bacterial spores.

As used herein, the term “sterilization” refers to a process that destroys or eliminates all forms of microbial life, including bacterial spores.

As used herein, the term “ultraviolet (UV) light” refers to electromagnetic radiation within a wavelength range of approximately 10 to 400 nanometers, which includes UVA (315-400 nm), UVB (280-315 nm), and UVC (100-280 nm), wherein UVC radiation is particularly useful for germicidal applications and may be generated using UV-C LEDs, mercury lamps, or equivalent sources.

As used herein, the term “ultrasound machine,” refers to a medical imaging apparatus that utilizes acoustic energy (ultrasound waves) to visualize tissues, organs, or other internal features of a subject. An ultrasound machine typically comprises a main housing or console that contains electronic circuitry for generating, transmitting, and receiving ultrasound signals; a processor configured to reconstruct image data from received echo signals; and a display configured to present such image data in real time. The ultrasound machine can include user interface components such as control buttons, a keyboard, or a touchscreen to adjust imaging parameters, as well as one or more probe connectors that permit electrical communication with external ultrasound transducers (e.g., external probes, intracavitary probes, endoprobes, etc.). In certain configurations, the ultrasound machine may be a cart-mounted system, a desktop console, or a portable or handheld unit, and may include additional features such as a base, support stand, power supply, and network interfaces for data storage or transmission.

As used herein, the term “container” refers to any housing, enclosure, or compartment configured to receive at least a portion of a medical probe for disinfection.

As used herein, the term “cap” refers to a component configured to close or seal an opening of a container to contain UV light and prevent exposure to users during disinfection.

As used herein, the term “sensor” or “meter” refers to any instrument or device configured to detect, measure, or estimate a condition related to disinfection, such as UV exposure time, light intensity, or dosage, and may include digital timers, UV dosimeters, or optical sensors.

As used herein, the term “reader” refers to a component configured to obtain or detect information from an indicator associated with a medical device or probe, such as a barcode, RFID chip, or electronic identifier.

As used herein, the term “indicator” refers to a visible, machine-readable, or electronically detectable feature associated with a medical probe that may store information such as identification, disinfection status, or usage history.

As used herein, the terms “approximately,” “about,” or “around” generally mean within an acceptable range of typical measurement error or tolerance known in the art, such as plus or minus ten percent, unless otherwise specified.

As used herein, the term “comprising” is inclusive and open-ended, meaning that the system or method may include additional, unrecited elements or steps.

As used herein, the articles “a” or “an” preceding an element mean one or more of that element unless specifically limited to a single instance. It will be understood by those of ordinary skill in the art that, throughout this specification, the term “a” preceding an element encompasses both singular and plural embodiments unless the context clearly dictates otherwise. Likewise, the term “comprising,” which is synonymous with “including,” “containing,” or “characterized by,” is inclusive or open-ended and does not exclude additional, unrecited elements or steps. Other embodiments or implementations will be apparent to those skilled in the art from the teachings of the present description and therefore are not described in further detail herein.

Section titles and subtitles used throughout this disclosure are provided for the convenience of the reader and should not be construed as limiting the scope of the invention. Certain theories or mechanisms of action may be proposed or described herein; however, whether such theories are accurate or not shall not limit the scope of the invention, provided that the invention is practiced in accordance with the present disclosure. Any and all references cited throughout this specification are hereby incorporated by reference in their entirety for all purposes.

In the event of a conflict in terminology or definition, the definitions set forth in this document shall control.

In various embodiments, a medical device such as a medical probe including a transducer for intracavitary or endocavitary contact may undergo high-level disinfection and be used with a single-use protective transducer cover. The disinfection procedure, cover, or combination thereof must ensure protection against transmission of human pathogens, including, for example, human immunodeficiency virus (HIV), human papillomavirus (HPV), and hepatitis B virus.

Medical devices such as intracavitary or endocavitary probes including transvaginal, transrectal, intraoperative, and transesophageal probes require high-level disinfection before reuse on another patient. Chemical disinfectants are commonly used for this purpose. However, chemical-based disinfection may not always provide complete efficacy due to human error or limitations of the process. For example, depending on the chosen disinfectant, a probe may need to be thoroughly rinsed and dried in accordance with manufacturer guidelines. High-level chemical disinfectants often require clean and dry surfaces, as moisture may dilute the agent. Furthermore, precautions must be taken to protect workers and patients from the potential toxicity of such chemicals. Some disinfectants require immersion of the probe in liquid solutions, necessitating that the probe be submersible. Even after proper chemical disinfection, stored probes may become recontaminated before subsequent use. Studies have shown that intracavitary or endocavitary probes can remain contaminated despite high-level disinfection or use of a probe cover, and may still harbor pathogens such as HPV if not properly treated between uses.

In various embodiments, a device and related system for disinfecting medical probes is provided. The device may enhance patient safety by reducing contamination risk and allowing a probe to remain in a disinfected and protected state until subsequent use, thereby minimizing recontamination.

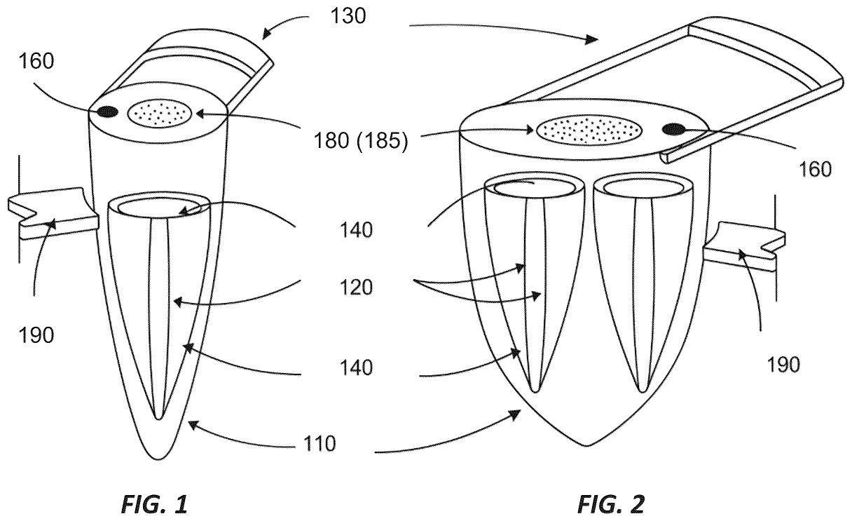

As illustrated in FIG. 1, a device 100 for disinfecting a medical probe may include a plurality of ultraviolet (UV) light emitters 120 configured to emit UV light within a container 110 to disinfect the probe surface.

The container 110 may include rigid and/or flexible portions. For example, the rigid portion can provide mechanical protection, while the flexible portion may accommodate probes of various shapes or sizes. The container may be constructed of UV-resistant materials such as polycarbonate or silicone rubber.

The container 110 may have an opening for insertion of at least a portion of the probe. The container can take various shapes such as cylindrical, bar, conical, or funnel-shaped to facilitate probe insertion. For instance, the cross-sectional area near the opening may be larger than areas further along its length to provide an expanded entry region.

The device 100 may include an operating cap 130 configured to close the opening of the container 110 and block UV light from escaping. The cap may include a cable-accommodating portion to enclose the probe cable and prevent leakage of UV light.

The cap 130 may incorporate seals or linings to eliminate gaps, including flexible rubber seals that conform around the cable. The cap may lock securely when closed and may activate or deactivate UV light emission based on its open or closed state, ensuring user safety.

In certain embodiments, closing the cap may automatically initiate a timed disinfection cycle controlled by a power timer. Upon completion, the UV light deactivates, and an indicator such as a signal light may notify the user that disinfection is complete and the probe remains sealed against recontamination.

Each UV emitter 120 may include a UV light source such as a bulb, fluorescent tube, or LED strip. The emitters may incorporate coatings or fluorescent materials to adjust emission wavelengths. Lenses or dispersers may be provided to focus or diffuse the UV light depending on probe geometry and distance.

In one embodiment, the device interior is lined with four equidistant UV-C LED strips to provide 360-degree exposure. The active disinfection zone may have a length of approximately 42.5 cm and a maximum width of approximately 15 cm.

A probe holder 140 may position the probe at an optimal distance from the UV emitters to achieve uniform light dispersion.

A meter 160 may measure exposure level, using a sensor, dosimeter, or timer. The meter may be fixed or mobile and located at a region of relatively lower UV intensity, such as the bottom of the container or the inner side of the cap. A UV-C dosimeter card may provide visual confirmation of successful disinfection.

The device 100 may include a reader to detect indicators associated with individual probes. Such indicators may include labels (barcodes or QR codes), RFID chips, or other electronic identifiers storing data such as probe ID, disinfection status, and time.

For example, a barcode scanner may log each probe identifier, the date and time of disinfection, and confirmation from the meter 160 of successful disinfection. This electronic record-keeping feature automates compliance documentation and replaces manual logging procedures.

The UV light may include UVA, UVB, UVC, or combinations thereof, typically within a wavelength range of about 10 nm to about 400 nm.

The device 100 may further include a vent 180 and fan to evacuate air from the container. A filter 185, such as an activated carbon filter, may remove odor or by-products like ozone generated by UV exposure.

A mounting bracket 190 may attach the device to a support structure such as a mobile caster pole.

In some embodiments, multiple probe holders and corresponding UV emitters may be provided within a single container to disinfect multiple probes simultaneously, as shown in FIG. 2.

A method of disinfecting a medical probe may include inserting at least a portion of the probe through an opening into the container 110, positioning it within a probe holder 140, and supplying power to the UV emitters 120 to irradiate and disinfect the probe surface.

The method may further include measuring UV exposure using the meter 160, venting air through the vent 180, and filtering exhaust air via the filter 185. The UV light may comprise UVA, UVB, UVC, or any suitable combination thereof.

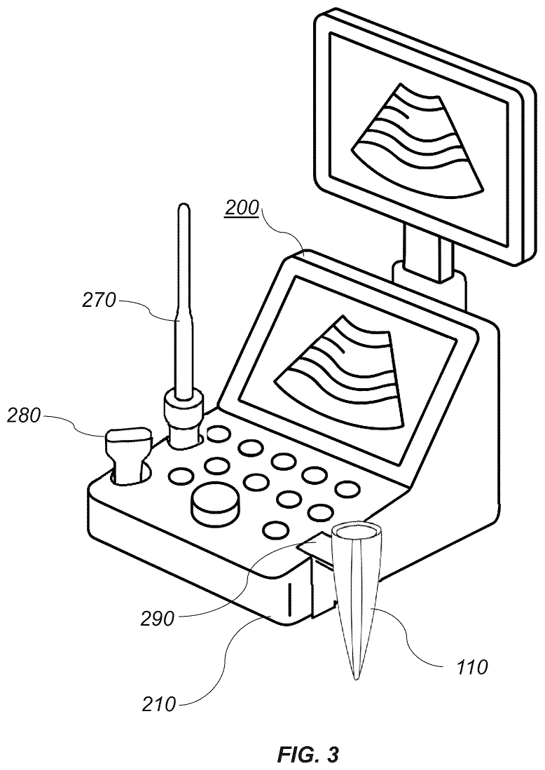

Referring to FIG. 3, a perspective view of an exemplary configuration is depicted in which a disinfection system is coupled to a medical device, here shown as an ultrasound system 200. Exemplary system 200 can include device controls and one or more interfaces for visualization and workflow, such as buttons for user instructions and at least one display interface. System 200 can include one or more recesses or receptacles configured to cradle probes that are not being disinfected or otherwise between uses. For example, in FIG. 3, an external probe 280 and endoprobe 270 are shown positioned within corresponding recesses or receivers of system 200 opposite edge 210. This arrangement allows the clinician to manage multiple probes in parallel: for example, one probe may be undergoing a disinfection cycle within container 110 while one or more additional probes are staged in other receptacles for system 200 for immediate availability.

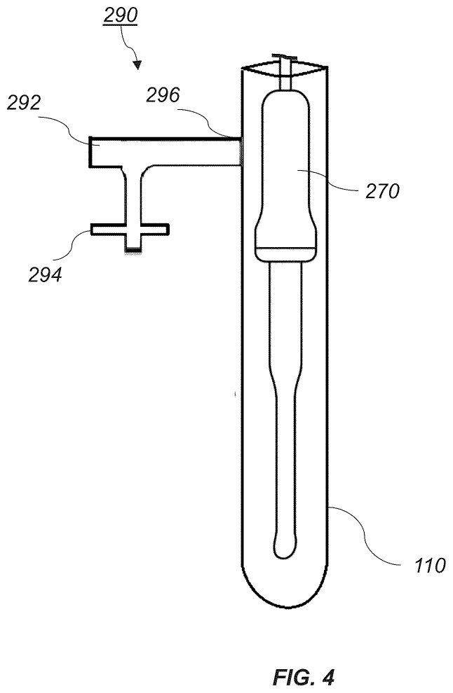

Container 110 as shown is mounted at a forward edge 210 of system 200 by an example bracket system 290. In the illustrated example, the container 110 is oriented such that its opening is accessible to a clinician positioned at the user interface side of system 200. In turn, a clinician can load and unload a probe without requiring the user to change position or obstruct access to other features of the ultrasound machine 200. Referring to FIG. 4, a side profile of container 110 coupled to system 290 is shown. In the illustrated view, the bracket system 290 is presented in isolation from system 200 strictly for illustrative purposes, to illustrate the structural relationships between the bracket components and container 110. System 290 includes a bracket 292 a clamp-type interface. In some embodiments, the bracket 292 includes opposed jaws or clamping surfaces, including a lower clamp surface 294 (e.g., so that surface 294 can engage a side of edge 210). In turn, system 290 can provide a stable, non-marring attachment to a corresponding system, such as system 200. Bracket 292 can incorporate a tightening mechanism, such as a threaded screw, lever-actuated cam, or quick-release fastener, to draw its clamping surfaces into frictional contact with an associated mounting surface (e.g., forward edge 210). In some aspects, system 290 includes a coupler portion extending from the clamp interface to the container 110, which may be implemented as a detachable or interchangeable connector 296 configured to engage a rail, beam, or groove structure of the container 110. In some examples, coupler 296 implements a sliding engagement between corresponding male and female profiles to facilitate rapid installation and removal, as shown more particularly herein, as well as positional adjustment to achieve a desired height or standoff relative to the corresponding system to which it is attached (e.g., system 200) and to establish a detachable, sliding engagement that enables tool-free installation, positional adjustment, and attachment of container 110 relative to system 290.

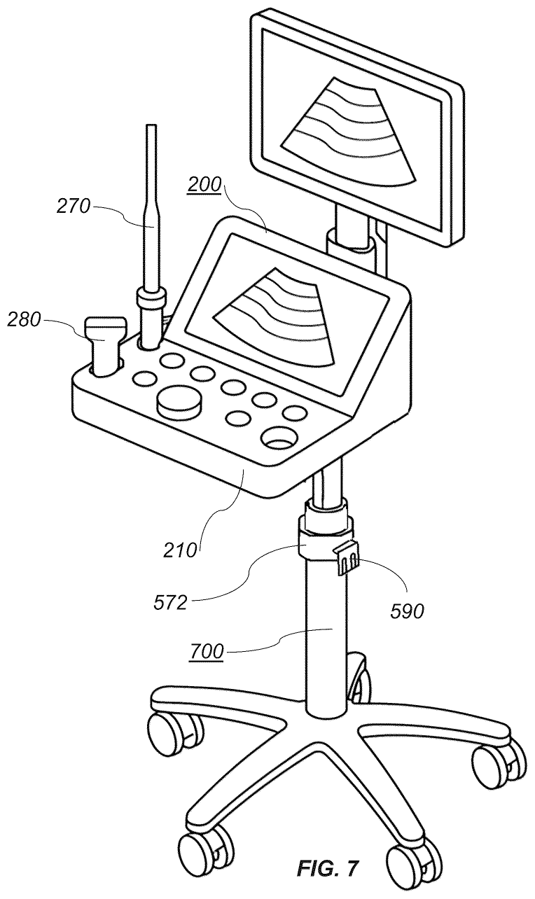

Although FIG. 3 illustrates container 110 mounted to system 200, system 290 can be configured to attach to a wide variety of medical devices and support structures commonly encountered in hospital environments. For example, system 290 can couple container 110 to edges, rails, shelves, or accessory mounts of anesthesia carts, endoscopy towers, patient monitoring systems, infusion pump stands, ventilator carts, and other wheeled or stationary platforms that house or interface with medical probes, such as shown in the example of FIG. 7. By providing attachment to both fixed and mobile platforms, system 290 imparts convenient, mobile UVC disinfection at the point of care for a range of probes, including but not limited to ultrasound transducers, temperature probes, EKG lead sets, oximetry sensors, and other devices contacting intact skin or mucous membranes as appropriate for the required level of disinfection.

In operation, a user can position a probe within container 110 by inserting the probe into the probe holder 140. With bracket system 290 securing container 110 system 200, the user may initiate a disinfection cycle via controls on container 110 or through a connected interface, while maintaining unimpeded access to system 200 controls and display. The configuration shown in FIG. 3 thereby facilitates high-level or intermediate-level disinfection workflows, depending on probe type and protocol, and extends naturally to other clinical contexts where rapid, repeatable, and mobile UVC probe disinfection is beneficial.

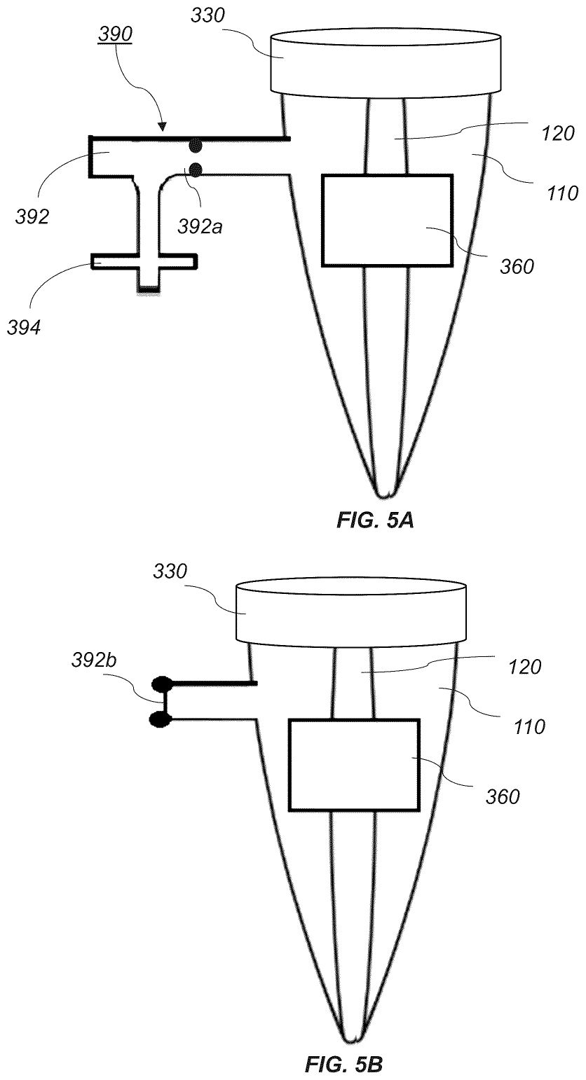

Referring to FIG. 5A, a side profile view of an exemplary container 110 coupled to another bracket system 390 is shown. System 390 includes a coupler assembly featuring complementary coupler elements 392a and 392b that establish a detachable, sliding connection between the bracket system 390 and the container 110. In the assembled state of FIG. 5A, the female coupler 392a is engaged with the male coupler 392b to secure the container 110 in a stable operating orientation. In various implementations, the coupler profiles can include a tongue-and-groove, dovetail, T-slot, or rectangular rail geometry configured to resist out-of-plane movement while enabling tool-free linear engagement and disengagement.

Container 110 illustrated in FIG. 5A includes an upper cap 330 that closes the probe-receiving opening. Cap 330 can be constructed from UV-blocking materials and may incorporate a perimeter gasket or seal to reduce or prevent UV light leakage during operation. Container 110 can include a display interface 360 positioned on an external surface of container 110, which can present information associated with current operations of the container 110, such as cycle initiation and completion, elapsed or remaining disinfection time, dose information from a meter 160, alert conditions, readiness status, and/or the like. Interface 360 can be oriented for visibility to a clinician standing at the equipment console or bedside, facilitating at-a-glance workflow.

FIG. 5B illustrates a partially disconnected configuration of the previous system 390 and container 110. Specifically, a distal portion of the male coupler 392b remains engaged with a corresponding interface on container 110, while the proximal portion of system 390 is separated such that the female coupler 392a is not shown. In various examples, the depicted arrangement of FIGS. 5A and 5B provides quick release for cleaning, transport, or reconfiguration between different devices and/or mounting locations, including edges or rails of medical devices or poles of mobile caster systems. The linear sliding path defined between the coupler features 392a and 392b can be arranged to allow transition between disengaged and secured positions while maintaining alignment and preventing unintended decoupling during normal operation.

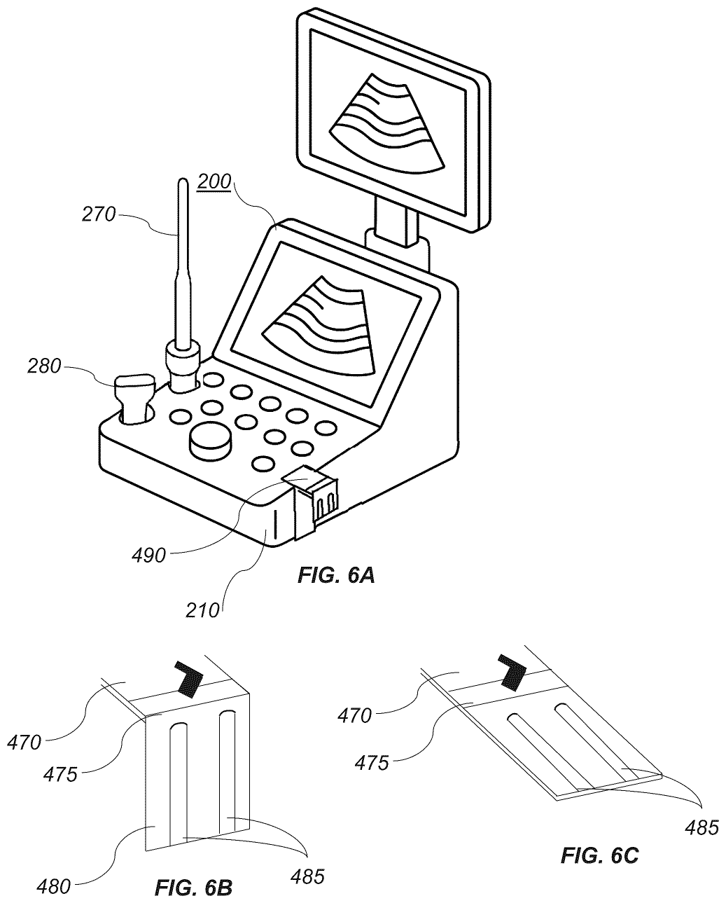

Referring to FIG. 6A, a perspective view is shown in which container 110 is mounted at forward edge 210 of system 200 by bracket system 490. Except for the bracket architecture, the arrangement is similar to that described with respect to FIG. 3. System 490 includes a proximal portion 470 configured to couple to the host device, here edge 210 of system 200, and a distal portion 480 configured to couple to container 110. A hinge 475 mechanically connects the proximal portion 470 to the distal portion 480 and permits controlled pivoting between portions. In some embodiments, the hinge 475 is implemented as a pin joint, a living hinge, a friction hinge, and/or a combination thereof, and can optionally include detents or adjustable friction to maintain a selected angular position. Portion 480 can include one or more female grooves 485 arranged to receive a complementary coupler, such as coupler 392b of the example depicted in FIGS. 5A and 5B.

Turning to FIG. 6B, a close-up partial view of system 490 is depicted in a disconnected, unhinged state. As shown, portion 480 is oriented angled downward and/or substantially orthogonal relative to portion 470. As shown, system 490 articulate via hinge 475, which allows distal portion 480 to pivot away from proximal portion 470 to facilitate loading and unloading of the container 110, to avoid interference with device controls or adjacent accessories, and/or to align grooves 485 with a mating coupler associated with container 110 during attachment. Grooves 485 are shown positioned at least partially within distal portion 480 to provide a stable and repeatable rail interface that can slidably receive a coupler, such as coupler 392b. In some embodiments, grooves 485 can include a series of aligned channels (e.g., a dovetail, T-slot, or rectangular rail profile) dimensioned to resist out-of-plane motion while enabling linear sliding engagement.

FIG. 6C shows a similar close-up partial view with the bracket system 490 in a connected state in which portion 480 and portion 470 are oriented substantially parallel or otherwise aligned. In this depicted orientation, grooves 485 are shown aligned to receive and retain the coupler of the container 110, such as the male coupler 392b, via a sliding motion that transitions from a disengaged position to a secured position. Hinge 475 permits pivoting into the substantially parallel orientation and, in some implementations, incorporates a lock, detent, friction element, or other retention feature to maintain the parallel relationship during disinfection. By localizing the grooves 485 predominantly within the distal portion 480, system 490 preserves consistent rail alignment with container 110 regardless of any variations in a mounting angle of portion 470 on a host device.

Referring to FIG. 7, an ultrasound system 200 is shown coupled to a mobile caster system 700. A bracket system 590, similar to system 490, is shown attached to a pole of system 700 by a coupler 572. Coupler 572 can be implemented as any device configured to detachably engage with the outer surface of a pole, such as a band clamp, split-ring collar, hinged clamp, lever-actuated cam clamp, or a quick-release fastener, and may include compliant or non-marring liners to enhance grip while protecting the pole surface. In some embodiments, coupler 572 can include an adjustable diameter and/or torque-limiting features to accommodate poles of different sizes while maintaining secure attachment during clinical use and transport.

In FIG. 7, container 110 is omitted to emphasize the mounting architecture of system 590 with respect to system 700 with bracket system 590 depicted in a disconnected, unhinged state with its distal portion oriented angled downward or substantially orthogonal relative to the proximal portion. This orientation facilitates clearance around adjacent equipment surfaces, enables ergonomic loading and unloading when the container 110 is present, and assists with alignment for sliding engagement with a compatible coupler on the container 110. Use of system 590 with the mobile caster system 700 enables point-of-care mobility for repositioning between rooms and bedside locations.

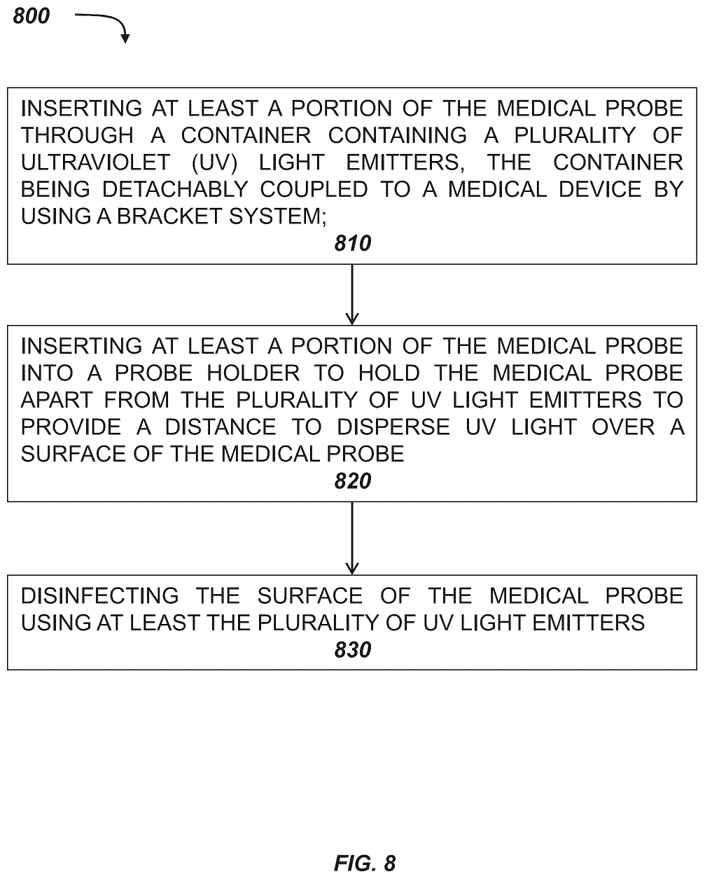

FIG. 8 is a flow diagram for a method 800 of disinfecting a medical probe according examples of this disclosure. Step 810 of method 800 can include inserting at least a portion of the medical probe through a container containing a plurality of ultraviolet (UV) light emitters, the container being detachably coupled to a medical device by using a bracket system. Step 820 of method 800 can include inserting at least a portion of the medical probe into a probe holder to hold the medical probe apart from the plurality of UV light emitters to provide a distance to disperse UV light over a surface of the medical probe. Step 830 of method 800 can include disinfecting the surface of the medical probe using at least the plurality of UV light emitters.

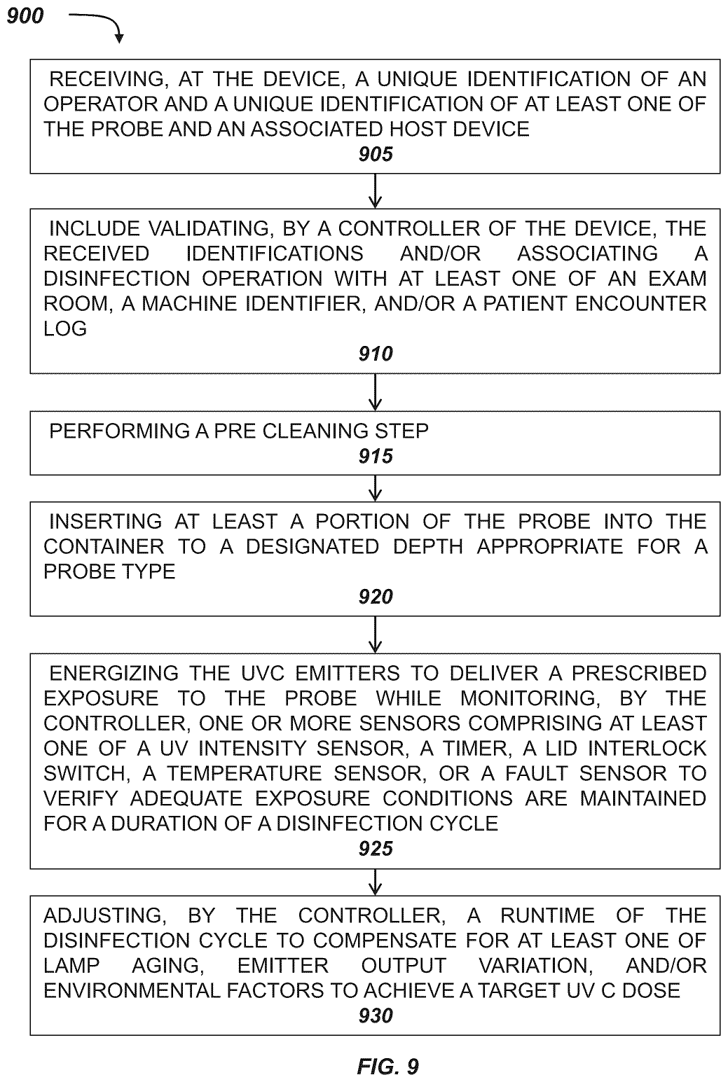

FIG. 9 is a flow diagram for a method of disinfecting a medical probe using a UV-C disinfection device that includes a container housing one or more UV-C emitters. Step 905 of method 900 can include receiving, at the device, a unique identification of an operator and a unique identification of at least one of the probe and an associated host device. In some aspects of step 905, the receiving can include at least one of manual entry via a user interface, selection from stored user profiles, and/or acquisition by scanning a machine-readable token that can include at least one of a barcode, a QR code, an RFID tag, etc. Step 910 of method 900 can include validating, by a controller of the device, the received identifications and/or associating a disinfection operation with at least one of an exam room, a machine identifier, and/or a patient encounter log. Step 915 of method 900 can include performing a pre-cleaning step. The pre-cleaning step can include chemically cleaning and/or wiping the probe with a compatible disinfectant to remove visible materials including one or more of gel, mucus, and blood so as to reduce bioburden and debris that could attenuate UV-C exposure. Step 920 of method 900 can include inserting at least a portion of the probe into the container to a designated depth appropriate for a probe type. In some examples of step 920, the inserting can be to at least one of depth markings, a probe holder, or internal guides that provide a mechanical stop or visual indication to ensure consistent positioning and spacing relative to the UV-C emitters. Step 925 of method 900 can include energizing the UV-C emitters to deliver a prescribed exposure to the probe while monitoring, by the controller, one or more sensors comprising at least one of a UV intensity sensor, a timer, a lid-interlock switch, a temperature sensor, or a fault sensor to verify adequate exposure conditions are maintained for a duration of a disinfection cycle.

In some aspects, method 900 can include adjusting, by the controller, a runtime of the disinfection cycle to compensate for at least one of lamp aging, emitter output variation, and/or environmental factors to achieve a target UV-C dose. Step 935 of method 900 can include detecting a safety condition violation and/or inadequate exposure and, in response, performing at least one of pausing the cycle, aborting the cycle, or prompting a user to take corrective action before resuming or restarting the cycle. In some aspects, method 900 can include upon successful completion of the disinfection cycle, automatically recording and time-stamping session data including at least one of the operator identification, the probe or device identification, start and end times, total exposure time, measured or estimated UV-C dose or intensity metrics, lid-interlock status, fault or warning codes, and cycle outcome. In some aspects, method 900 can include storing the session data in device memory and making the session data available for transmission to an external system. In some aspects, method 900 can include transmitting, in some implementations, the session data over at least one of a wired or wireless interface to update a centralized log used for at least one of quality control, traceability, or regulatory compliance, and indicating completion of the cycle to permit removal of the probe for subsequent use. In some aspects, method 900 can include routing a probe cord, if present, into a curved safety channel of the safety lid and closing the safety lid into an operating position to reduce or prevent UV-C radiation from escaping the container. In some aspects, method 900 can include actuating one or more interlock sensors upon closing of the safety lid to confirm at least proper sealing of the container and correct routing of the probe cord in the safety channel, and transitioning the device to an operating mode when a safety condition is satisfied.

It is intended that the appended claims cover all such modifications and applications that fall within the true scope of the invention. The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

What is claimed is:

1. A disinfection system for a medical probe, comprising:
a container configured to receive the medical probe;
a plurality of ultraviolet (UV) light emitters for emitting UV light in the container to disinfect a surface of the medical probe; and
a bracket system coupled to the container and configured to couple the container to a portion of a medical device comprising the medical probe or a mobile caster holding pole.

2. The system of claim 1, further comprising: a probe holder to hold the medical probe apart from the plurality of UV light emitters to provide a distance to disperse the UV light over the surface in the container when at least a portion the medical probe is inserted into the container and the probe holder, and
wherein the medical device comprises an ultrasound machine including a base and control housing configured to store the medical probe, the medical probe comprising one or more ultrasound probes, and wherein the bracket system is a detachable bracket configured to support the container and attach to an edge of the ultrasound machine.

3. The system of claim 2, wherein the ultrasound machine further comprises an endoprobe and an external probe each configured for selective connection to the ultrasound machine.

4. The system of claim 1, wherein the bracket system is configured to detachably attach to an edge of a base of the medical device.

5. The system of claim 4, wherein the bracket system includes a clamp mount comprising upper and lower opposed clamping surfaces configured to engage opposite sides of the edge of the base.

6. The system of claim 4, wherein the bracket system includes a mechanical coupler at an opposite end configured to support or connect to the container.

7. The system of claim 6, wherein the mechanical coupler comprises an articulating or pivoting joint configured to adjust an orientation of the receptacle relative to the bracket system.

8. The system of claim 1, wherein the pole includes a caster-mounted base and a central vertical rod, and the bracket system includes a clamp configured to engage an outer surface of the pole, wherein the clamp includes a tightening mechanism selected from the group consisting of: a threaded screw, a lever-actuated cam, and a quick-release fastener.

9. The system of claim 1, wherein the bracket system and the container is configured to support an endoprobe simultaneous with a receptacle of the medical device supporting one or more other probes.

10. The system of claim 1, wherein the bracket system comprises a multi-part bracket assembly including a first bracket member having at least one female groove and a counterpart second bracket member having at least one corresponding male groove configured to slide into and out of the female groove to provide a detachable sliding engagement.

11. The system of claim 10, wherein the male groove is formed on a beam extending outwardly from an outer surface of the container wherein the beam is detachably connectable to the outer surface of the container, and wherein the beam includes a mounting interface comprising at least one a keyed slot, a dovetail, and a fastener-receiving boss configured to secure the beam to the outer surface of the container.

12. The system of claim 1, wherein the bracket system comprises a proximal portion pivotably connected to a distal portion.

13. The system of claim 12, wherein, in an unconnected configuration, the distal portion is oriented angled downward relative to the proximal portion, and, wherein, in a connected configuration, the distal portion is pivotable to an orientation in which the proximal and distal portions are substantially parallel.

14. The system of claim 12, wherein the distal portion includes one or more male grooves configured to detachably connect with corresponding female grooves of a beam extending from the container.

15. The system of claim 12, wherein the distal portion includes one or more female grooves configured to detachably connect with corresponding male grooves of a beam extending from the container.

16. The system of claim 15, wherein the one or more male grooves are configured to permit the beam to slide into and out of secure engagement with the distal portion to facilitate coupling and decoupling of the container to the medical device, and wherein the one or more female grooves are configured to permit the beam to slide into and out of secure engagement with the distal portion to facilitate coupling and decoupling of the container to the medical device.

17. A method of disinfecting a medical probe, comprising:
inserting at least a portion of the medical probe through a container containing a plurality of ultraviolet (UV) light emitters, the container being detachably coupled to a medical device by using a bracket system;
inserting at least a portion of the medical probe into a probe holder to hold the medical probe apart from the plurality of UV light emitters to provide a distance to disperse UV light over a surface of the medical probe; and
disinfecting the surface of the medical probe using at least the plurality of UV light emitters.

18. The method of claim 17, wherein the bracket system comprises a first bracket member with at least one female groove and a second bracket member with at least one corresponding male groove, the method further comprising: detachably coupling the container to the medical device by sliding the male groove into the female groove.

19. The method of claim 17, further comprising: pivoting a distal portion of the bracket system relative to a proximal portion of the bracket system wherein pivoting the distal portion includes, in a connected configuration, pivoting the distal portion to an orientation in which the proximal and distal portions are substantially parallel.

20. A bracket system configured to detachably mount a container to a medical device or a mobile caster holding pole, the bracket system comprising:
a proximal portion including a mounting interface configured to couple to the medical device or the pole;
a distal portion pivotally connected to and/or extended from the proximal portion, the distal portion including a container interface configured to couple to the container.
